(19) United States
(12) Patent Application Publication
(10) Pub. No.: US 2007/0027153 A1
(43) Pub. Date: Feb. 1, 2007

(54) Topical skin-protectant and anti-pruritic compositions

(76) Inventors: Kevin M. Reeth, Cumming, GA (US); Jeffrey S. Reynolds, Greenville, NY (US); Kathleen L. Clark, Medusa, NY (US)

Correspondence Address: NATH & ASSOCIATES, 112 South West Street, Alexandria, VA, 22314, US

(21) Appl. No.: 11/190,116

(22) Filed: Jul. 27, 2005

(51) Int. Cl. A61K 31/5375; A61K 8/81

(52) U.S. Cl. 514/238.2; 424/70.16

(57) ABSTRACT

Topical skin protectant compositions, and more particularly anti-pruritic skin protectant compositions, comprising a skin protective ingredient, a therapeutically effective amount of a pharmaceutically active agent comprising an anesthetic agent or derivative thereof, an oleaginous solvent comprising a substance other than the skin protective ingredient, and an aqueous solvent. These skin protectant compositions are capable of temporarily or permanently reducing, inhibiting, treating, ameliorating, or preventing pruritic skin conditions, as well as other related skin conditions. These compositions are further capable of restoring or repairing a skin lipid barrier of a mammal.

### FIELD OF THE INVENTION

The present subject matter relates generally to topical skin protectant compositions, and more particularly to anti-pruritic skin protectant compositions, comprising a skin protective ingredient, a therapeutically effective amount of a pharmaceutically active agent comprising an anesthetic agent or derivative thereof, an oleaginous solvent comprising a substance other than the skin protective ingredient, and an aqueous solvent. These skin protectant compositions are capable of temporarily or permanently reducing, inhibiting, treating, ameliorating, or preventing pruritic skin conditions, as well as other related skin conditions. These compositions are further capable of restoring or repairing a skin lipid barrier of a mammal.

### BACKGROUND OF THE INVENTION

The skin is the largest organ of the body and serves as a barrier protecting mammalian organisms from both aqueous and xerotic ambient environments. The maintenance of this barrier against excessive transcutaneous water loss to the environment is critical for survival of all terrestrial animals. In mammals, this barrier is formed by the anucleate, cornified, outermost layers of the epidermis, collectively known as the stratum corneum.

Mammalian skin has a tendency to dry out when exposed to low humidity or to harsh detergent solutions for extended periods of time, causing pruritus. From a physiological standpoint, such dryness is a measure of the water content of the skin. Under normal conditions, the vapor pressure and water content of mammalian skin are typically higher than that of the air surrounding the skin, which ultimately results in evaporation of water from the skin's surface. Skin becomes dry due to an excess loss of water, in particular the loss of water from the stratum corneum. Low humidity speeds up this process, exacerbating the drying of skin. Also, continuous and prolonged contact with or immersion in soap or detergent solutions can contribute to dryness of the stratum corneum. Typically, these solutions promote dissolution of the skin surface and lipid layers, as well as the dissolution of the hygroscopic water-soluble components of the skin.

Excessive water loss from the stratum corneum can lead to dry, flaky, scaly, and/or itchy skin. Such excessive water loss can also lead to excessive peeling or flaking of the outermost keratinaceous material of the stratum corneum (i.e. excessive desquamation). If excessive water loss from the stratum corneum continues, skin can crack and become excessively itchy and irritated, which can lead to inflammation and even infection.

In normal skin, the stratum corneum is shed as individual cells or as small clusters of cells. Skin conditions such as pruritus, dry skin, psoriasis, ichthyosis, dandruff, acne, callus, photodamaged skin, aged skin, and sunburn can be described as disorders of keratinization in which the shedding of stratum corneum cells at the skin surface is altered relative to normal, young, healthy skin. Such alteration instead results in shedding of large clusters of cells, potentially leading to visible scaling of the skin, a build-up of keratinaceous material on the skin surface or in skin follicles or ducts, and/or a rough texture to the skin surface. Many times these conditions are due, at least in part, to the reduced amount of water available to the skin, in particular to the reduced amount of water available to the stratum corneum. Accordingly, these conditions can sometimes be improved by desquamation.

However, desquamation is not always effective in reducing, inhibiting, treating, preventing, or ameliorating these conditions, in particular pruritus, since excessive desquamation can itself cause pruritic skin conditions. Instead of desquamation, lubricants can be used in an attempt to alleviate these skin conditions. In normal skin, sebaceous glands secrete sebum, which is a complex mix of triglycerides, waxes, cholesterol, and esters with mild anti-bacterial and anti-fungal activity. Sebum naturally helps lubricate the skin, which enables the skin to retain water intercellularly within the stratum corneum layer, and generally improves the look and feel of skin. Conversely, in unhealthy or abnormal skin, sebum is often produced at insufficient levels to help alleviate a pruritic skin condition, let alone improve the look and feel of the skin.

Accordingly, many topical formulations have been devised in an attempt to increase the oleaginous liquids available to aid in retaining water intercellularly within the stratum corneum, and relieve or cure pruritic skin conditions, as well as improve the look and feel of skin. These topical formulations typically comprise a skin protective ingredient in an oil-in-water emulsion or water-in-oil emulsion, with the skin protective ingredient usually having an oil constituent comprising at least one long chain hydrocarbon, or a similar component. Petrolatum, for example, and other similar oils and liquid or semi-liquid, hydrophobic, hydrocarbon-based components have been used as skin protective ingredients in topical formulations for a variety of purposes. When used to treat pruritic skin conditions, petrolatum can be very effective. However, due to petrolatum's relatively unstable nature and hydrophobic qualities, it is often difficult to incorporate petrolatum into aqueous-containing, topical formulations in high quantities. In this regard, skin protective ingredients in general can be relatively unstable, especially in emulsions or dispersions.

For example, U.S. Pat. No. 5,607,980 to McAtee et al. discloses compositions for improving skin feel, conditioning, desquamating, cleansing skin, and relieving dry skin. In particular, McAtee discloses topical personal care compositions having an amphoteric surfactant in the amount of about 0.1% to about 20% by weight, an anionic surfactant in the amount of about 0.1% to about 20% by weight, a cationic surfactant in the amount of about 0.1% to about 15% by weight, and water in the amount of about 45% to about 99.7% by weight.

Additionally, several previous topical formulations have been prepared containing a topical anesthetic, in addition to these oleaginous components, in an attempt to help relieve mild to moderate pain associated with various skin disorders. Typically, these anesthetics are present in low to moderate dosages and comprise the active ingredient of the formulation.

In this regard, U.S. Pat. Nos. 5,665,364 and 5,811,111 to McAtee et al. disclose compositions for the delivery of active agents. The disclosed compositions have about 0.1% to about 20% by weight of an amphoteric surfactant, about 0.1% to about 20% by weight of an anionic surfactant, about 0.001% to about 20% of an active ingredient, and about 40% to about 99.799% by weight of water. The compositions are disclosed as useful for treating conditions such as acne, skin lesions, blemishes, and other imperfections. The compositions are also disclosed as nonirritating to the skin and providing improved skin feel benefits. However, these patents do not disclose compositions containing a high level of a skin protective ingredient effective to both treat pruritus and repair the skin lipid barrier.

Likewise, U.S. Pat. No. 5,961,997 to Swinehart discloses anti-pruritic compositions containing menthol, camphor, and phenol in a carrier. Swinehart discloses that the compositions preferably further comprise topical anesthetics such as lidocaine and pramoxine, and more preferably further comprise lidocaine, pramoxine, and hydrocortisone acetate. Swinehart additionally discloses that the compositions are oil-free, lanolin-free, fragrance-free, free of formaldehyde-releasing preservatives, hypoallergenic, noncomedogenic, and nonacnegenic. Moreover, Swinehart discloses that these compositions are capable of relieving itching in patients suffering from a variety of dermatoses or pruritus. However, Swinehart teaches away from compositions containing a skin protective ingredient in addition to the active ingredients.

Additionally, U.S. Pat. No. 6,214,318 to Osipow et al. discloses aerosol ointment compositions which can produce a sustained cooling effect that provides fast relief from pain and itching as well as a tendency to shrink swollen, inflamed tissue upon topical application. The disclosed compositions contain oils, thickening agents for the oils, a propellant, and a therapeutic agent. Again, however, Osipow et al. do not disclose the presence of a skin protective ingredient in the embodied compositions.

U.S. Pat. No. 6,699,488 to Deckner et al. further discloses rinsable skin conditioning compositions having high internal phase emulsions that are substantially free of a surfactant. In particular, the compositions have from about 20% to about 90% by weight of an oil, about 0.1% to about 10% by weight of a stabilizer, about 9.5% to about 79.5% by weight of water, and about 0% to about 2% by weight of a perfume. The disclosed compositions can deposit conditioning agents, benefit agents, and/or other conventional cosmetic or skin care ingredients on skin. However, compositions having such a high internal phase emulsion are often difficult to form and maintain as a stable composition.

Similarly, U.S. Patent Application Publication No. 2002/0034489 to Wiegland et al. discloses a method of depositing a benefit agent on a keratinous surface by applying a ringing gel composition comprising a surfactant phase, an oil phase, and a benefit agent. In particular, the proportion of oil in the ringing gel composition preferably ranges from about 5% to about 50%, more preferably from about 10% to about 35%, and most preferably from about 15% to about 25% by weight.

Accordingly, stable topical anti-pruritic compositions containing both an anesthetic agent and high levels of a skin protective ingredient were previously unknown in the art. Moreover, many of the previously known compositions were not recognized as capable of both treating a pruritic skin condition and repairing the skin lipid barrier.

For these reasons, there remains a need in the art for stable topical anti-pruritic compositions that are effective in temporarily or permanently reducing, inhibiting, treating, preventing, or ameliorating pruritic skin conditions. Additionally, there remains a need in the art for such compositions that are also capable of restoring or repairing a skin lipid barrier of a mammal. In this regard, there remains a need for stable topical anti-pruritic compositions comprising a high quantity of a skin protective ingredient in addition to an anesthetic agent. The present subject matter addresses these needs.

### SUMMARY OF THE INVENTION

The present subject matter relates generally to skin protectant compositions, and more particularly to topical anti-pruritic skin protectant compositions.

In this regard, a preferred embodiment of the present subject matter relates to a topical skin protectant composition comprising:

(i) a therapeutically effective amount of a pharmaceutically active agent comprising an anesthetic agent or a derivative thereof;

(ii) about 25 to about 65% by weight of a skin protective ingredient;

(iii) an oleaginous solvent comprising a substance other than said skin protective ingredient; and

(iv) an aqueous solvent.

Another preferred embodiment of the present subject matter relates to a topical emulsion composition comprising:

(i) about 0.001 to about 5% by weight of a pharmaceutically active agent comprising an anesthetic agent or a derivative thereof;

(ii) about 25 to about 65% by weight of a skin protective ingredient;

(iii) about 0.1 to about 10% by weight of an anti-pruritic agent;

(iv) at least about 8% by weight of an oleaginous solvent comprising a substance other than said skin protective ingredient; and

(v) about 35 to about 75% by weight of an aqueous solvent.

Yet another preferred embodiment of the present subject matter relates to a method for inhibiting or treating a pruritic skin condition in a patient, which comprises administering to a patient in need thereof a topical composition comprising:

(i) a therapeutically effective amount of a pharmaceutically active agent comprising an anesthetic agent or a derivative thereof;

(ii) about 25 to about 65% by weight of a skin protective ingredient;

(iii) an oleaginous solvent comprising a substance other than said skin protective ingredient; and

(iv) an aqueous solvent;

wherein said skin protective ingredient enhances the ability of the pharmaceutically active agent to inhibit or treat the pruritic skin condition.

### DETAILED DESCRIPTION OF THE INVENTION

### Definitions

As used herein, the terms “administering”, “administration”, and like terms refer to any method which, in sound medical or cosmetic practice, delivers the composition to a subject in such a manner as to provide a positive effect on a dermatological disorder, condition, or appearance. The compositions are preferably administered such that they cover the entire area to be treated. “Direct administration” refers to any method which, in sound medical or cosmetic practice, delivers the composition to a subject without the use of another composition, delivery agent, or device. “Indirect administration” refers to any method which, in sound medical or cosmetic practice, delivers the composition to a subject with the use of at least another composition, delivery agent, or device.

As used herein, the phrases an “effective amount” or a “therapeutically effective amount” of a pharmaceutically active agent or ingredient, which are synonymous herein, refer to an amount of the pharmaceutically active agent sufficient enough to have a positive effect on the area of application. Accordingly, these amounts are sufficient to modify the skin disorder, condition, or appearance to be treated but low enough to avoid serious side effects, within the scope of sound medical or dermatological advice. A therapeutically effective amount of the pharmaceutically active agent will cause a substantial relief of symptoms when applied repeatedly over time. Effective amounts of the pharmaceutically active agent will vary with the particular condition or conditions being treated, the severity of the condition, the duration of the treatment, the specific components of the composition being used, and like factors.

As used herein, the phrase “oleaginous solvent” refers to a chemical ingredient or combination of chemical ingredients present in the instant compositions in which the majority of the collective ingredient(s) comprises a carbon structure and has at least one property of an oil.

As used herein, the phrase “pharmaceutically acceptable salts” refers to salts of certain ingredient(s) which possess the same activity as the unmodified compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Ethanolamine is particularly preferred in this regard. Further poorly volatile bases which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also by used, as can guanidine and its derivatives, in particular its alkylation products. However, it is also possible to employ as salt-forming agents, for example, low molecular weight alkylamines such as methylamine, ethylamine, or triethylamine. Suitable salts for the components to be employed according to the present subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

As used herein, the phrase “pruritic skin condition” refers to a condition in which at least an itchy sensation occurs on at least one skin area of a mammal.

As used herein, the phrase “skin protectant” refers to a composition or compositions that have the ability to repair interstitial lipid layers, provide lipid restoration, provide skin barrier restoration, increase water amounts intercellularly within at least one skin layer, and/or result in improvements in skin integrity.

As used herein, the phrase “therapeutic composition” refers to a composition which, upon administration, demonstrates a therapeutic affect upon a mammal.

Other terms as used herein are meant to be defined by their well-known meanings in the art.

### Topical Skin Protectant Compositions

A preferred aspect of the subject matter expressed herein relates to various topical skin protectant and anti-pruritic compositions. In this regard, the present subject matter preferably relates to a topical skin protectant composition comprising:

(i) a therapeutically effective amount of a pharmaceutically active agent comprising an anesthetic agent or a derivative thereof;

(ii) about 25 to about 65% by weight of a skin protective ingredient;

(iii) an oleaginous solvent comprising a substance other than said skin protective ingredient; and

(iv) an aqueous solvent.

Skin protective ingredients are generally used to temporarily or permanently alleviate, reduce, inhibit, treat, cure, or prevent skin disorders such as pruritus, dry skin, chapped skin, chafed skin, psoriasis, ichthyosis, dandruff, acne, callus, photodamaged skin, aged skin, sunburn, and similar disorders due to the advantageous properties of the skin protective ingredient. However, as previously discussed, compositions containing skin protective ingredients are generally unstable due to their volatile, flammable, and/or reactive properties. Accordingly, the presently preferred compositions are advantageous over previous compositions in that they maintain stability over time despite the presence of high amounts of a skin protective ingredient.

Additionally, skin protective ingredients usually express some degree of hydrophobicity, so their inclusion into a composition having an aqueous phase is difficult to accomplish without diminishing or inactivating their advantageous properties. However, the presently preferred compositions are unique in that they are formed as emulsions containing both an aqueous and oily phase, without diminishing the advantageous properties of the skin protective ingredient incorporated therein.

Moreover, many of the previously known compositions containing a skin protective ingredient also contained pharmaceutically active agents such as topical or local anesthetics to temporarily alleviate the mild or moderate irritation and pain associated with skin disorders and other disorders. However, these compositions were often unable to incorporate a therapeutically effective amount of the pharmaceutically active agent with a large amount of a skin protective ingredient into a single, stable composition for topical application without diminishing or inactivating the advantageous properties of the skin protective ingredient and pharmaceutically active agent.

In contrast, the preferred compositions herein are unique in that they combine a therapeutically effective amount of a pharmaceutically active agent and a large amount of a skin protective ingredient into a single topical composition without diminishing or inactivating the advantageous properties of either component. Further, these compositions combine these ingredients into a stable topical composition having a substantial aqueous phase. Accordingly, these compositions are advantageous over previous compositions that either contain lesser amounts of a skin protective ingredient, or are less stable and diminish the effectiveness of the individual components.

In this regard, the preferred topical pharmaceutical compositions are further unique in that they are storage stable with respect to both the skin protective ingredient component and the pharmaceutically active ingredient. Accordingly, these compositions have a decided advantage over previous skin protective ingredient/pharmaceutically active ingredient compositions in that they limit the amount of degradation of these ingredients over time, resulting in a composition with improved long-term efficacy at temperatures of about 30° C. or below. In this regard, the present compositions are preferably able to maintain a purity of at least 90% and a concentration of degradation product(s) less than about 10% of the starting concentration of the pharmaceutically active ingredient.

Further, the remarkable stability of the preferred compositions solves long felt difficulties in formulating skin protective ingredient/pharmaceutically active ingredient compositions. Since these compositions have an increased stability over similar compositions previously known in the art, they provide unexpected advantages over the prior art compositions. For example, the increased storage stability permits the presently preferred compositions to be manufactured in greater quantities without fear that the compositions produced will be wasted. Further, the enhanced stability provides the presently preferred compositions with an enhanced effect in treating skin disorders treatable with a skin protective ingredient and/or an anesthetic agent over the previously known compositions.

The selection of specific excipients and amounts thereof in the presently preferred compositions, as well as the preparation of compositions having a specific designated pH in the form of a designated emulsion, conveys these unique stability characteristics to the presently preferred compositions.

Anesthetic Agent

An essential component of the preferred compositions is a pharmaceutically active agent comprising an anesthetic agent or a derivative thereof. The anesthetic agent provides minor to moderate pain relief and helps alleviate itchy, burning, and/or irritated sensations caused by various skin disorders, such as pruritus. The anesthetic agent is preferably present in the instant compositions in a therapeutically effective amount. In this regard, the present compositions preferably contain about 0.01% to about 20% by weight, and more preferably from about 0.01% to about 5% by weight, of the anesthetic agent.

Non-limiting examples of preferred anesthetic agents useful herein include pramoxine, diphenhydramine, benzocaine, lidocaine, bupivacaine, chloroprocaine, dibucaine, etidocaine, mepivacaine, tetracaine, dyclonine, hexylcaine, lignocaine, phenacaine, procaine, ketamine, phenol, butamben, butambenpicrate, cocaine, dimethisoquin, diperodon, dyclonine, methapyriline, oxyprocaine, p-buthylaminobenzoic acid 2-(die-ethylamino) ethyl ester, piperocaine, prilocaine, tripelennamine, benzyl benzoate, calamine, chloroxylenol, dyclonine, resorcinol, troclosan, cinchocaine, dexivacaine, diamocaine, levobupivacaine, oxethazaine, proparacaine, propoxycaine, pyrrocaine, risocaine, rodocaine, ropivacaine, pramocaine, proxazocain, 4-(3-(p-butoxyphenoxy)propyl) morpholine, gamma-morpholinopropyl 4-n-butoxyphenyl ether, p-butoxyphenyl gamma-morpholinopropyl ether, 4-[3-(4-butoxyphenoxy)-propyl]morpholine, pharmaceutically acceptable salts thereof, and mixtures thereof.

In a particularly preferred embodiment, the anesthetic agent is pramoxine or a pharmaceutically acceptable salt thereof.

Anti-pruritic Agent

In a preferred embodiment, the present compositions can optionally further comprise about 0.1% to about 10% by weight of an anti-pruritic agent. This anti-pruritic agent can enhance the effectiveness of the present compositions in treating pruritus, as well as in providing skin protection.

Preferred, non-limiting examples of anti-pruritic agents useful in this regard include menthol, camphor, phenol, methyl anthranilate, menthyl anthranilate, derivatives thereof, and mixtures thereof. Menthol and menthol derivatives are particularly preferred in this regard.

Optional Additional Active Agent

In further, alternative embodiments of the present subject matter, the present compositions can optionally comprise another pharmaceutically active agent in addition to the anesthetic agent. Preferred, non-limiting examples of such additional pharmaceutically active agents include flavonoids, anti-cellulite agents, anti-inflammatory agents, tanning agents, anti-microbial agents, anti-fungal agents, sunscreens, anti-wrinkle agents, anti-atrophy agents, anti-acne agents, and mixtures and combinations thereof.

Non-limiting examples of preferred flavonoids useful in this regard include unsubstituted flavanone, mono-hydroxy flavanones, mono-alkoxy flavanones, unsubstituted chalcone, mono-hydroxy chalcones, di-hydroxy chalcones, tri-hydroxy chalcones, unsubstituted flavone, 2′,3′,4′-trihydroxy chalcone, 4,2′,4′-trihydroxy chalcone, 2,2′,4′-trihydroxy chalcone, 2′,4-dihydroxy chalcone, 2′,4′-dihydroxy chalcone, 2,2′-dihydroxy chalcone, 2′,3-dihydroxy chalcone, 2′,5′-dihydroxy chalcone, 2′-hydroxy chalcone, 4′-hydroxy chalcone, 5-methoxy flavanone, 6-methoxy flavanone, 7-methoxy flavanone, 4′-methoxy flavanone, 2′-hydroxy flavanone, 6-hydroxy flavanone, 7-hydroxy flavanone, 7,2′-dihydroxy flavone, 3′,4′-dihydroxy naphthoflavone, 4′-hydroxy flavone, 5,6-benzoflavone, 7,8-benzoflavone, unsubstituted isoflavone, daidzein, 7,4′-dihydroxy isoflavone, 5,7-dihydroxy-4′-methoxy isoflavone, soy isoflavones, unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, apigenin glycoside, luteolin glycoside, 6-methoxy-quercetin-glycoside, quercetin, luteolin, pharmaceutically acceptable salts thereof, and mixtures thereof.

Non-limiting examples of preferred anti-cellulite agents useful in this regard include xanthine compounds, caffeine, theophylline, theobromine, aminophylline, vexel, cyclolipase, coaxel, Pleurimincyl, Lipocare® available from Lipo Chemicals, Inc., Paterson, N.J., unislim, pharmaceutically acceptable salts thereof, and mixtures thereof.

Non-limiting examples of preferred anti-inflammatory agents useful in this regard include propionic acid derivatives, acetic acid derivatives, fenamic acid derivatives, biphenylcarboxylic acid derivatives, oxicams, acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, microprofen, tioxaprofen, suprofen, alminoprofen, tiaprofenic acid, fluprofen, bucloxic acid, apazone, bromfenac, celecoxib, diclofenac, difenpiramide, diflunisal, etodolac, flufenamic acid, indomethacin, ketorolac, meclofenamate, mefenamic acid, meloxicam, nabumetone, phenylbutazone, piroxicam, butibufen, rofecoxib, salicylic acid, sulindac, tolmetin, ketorolac tromethamine, antihistaminic agents, diphenhydramine, chlorpheniramine, diphenhydramine hydrochloride, chlorpheniramine maleate, corticosteroids, alclometasone, dexamethasone, flumethasone, hydrocortisone, hydrocortisone-21-monoesters, hydrocortisone-21-acetate, hydrocortisone-21-butyrate, hydrocortisone-21-propionate, hydrocortisone-21-valerate, hydrocortisone-17,21-diesters, hydrocortisone-17,21-diacetate, hydrocortisone-17-acetate-21-butyrate, hydrocortisone-17,21-dibutyrate, prednisolone, methylprednisolone, betamethasone benzoate, betamethasone diproprionate, clobetasol propionate, diflorasone diacetate, fluocinonide, fluticasone propionate, mometasone furoate, triamcinolone acetonide, topical corticosteroids, hydroxyltriamcinolone, alpha-methyl dexamethasone, dexamethasone-phosphate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, medrysone, amcinafel, amcinafide, betamethasone, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisone, beclomethasone dipropionate, triamcinolone, isoxicam, tenoxicam, sudoxicam, CP-14, 304, salicylates, disalcid, benorylate, trilisate, safapryn, solprin, fendosal, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acid, pyrazoles, phenylbutazone, oxyphenbutazone, feprazone, azapropazone, trimethazone, candelilla wax, bisabolol, alpha bisabolol, aloe vera, plant sterols, phytosterol, Manjistha, Guggal, kola extract, chamomile, red clover extract, Piper methysticum extract, Bacopa monieri extract, sea whip extract, licorice, glycyrrhetic acid, glycyrrhizic acid, monoammonium glycyrrhizinate, monopotassium glycyrrhizinate, dipotassium glycyrrhizinate, 1-beta-glycyrrhetic acid, stearyl glycyrrhetinate, 3-stearyloxy-glycyrrhetinic acid, disodium 3-succinyloxy-beta-glycyrr-hetinate, stearyl glycyrrhetinate, pharmaceutically acceptable salts thereof, and mixtures thereof.

Non-limiting examples of preferred tanning agents useful in this regard include dihydroxyacetone, tyrosine, ethyl tyrosinate, phospho-DOPA, pharmaceutically acceptable salts thereof, and mixtures thereof.

Non-limiting examples of preferred anti-microbial and anti-fungal agents useful in this regard include beta-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, zinc erythromycin, erythromycin estolate, erythromycin steaerate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mendelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xyleneol, nystatin, tolnaftate, clotrimazole, benzoyl peroxide, azelaic acid, ethyl acetate, meclocycline, lincomycinics, tetracyclinics, sulfur-based antibiotics, sulfonamides, mupirocin, magainin I, magainin II, lincomycin, (6,8-dideoxy-6-[[(1-methyl-4-propyl-2-pyrrolidinyl)-carbonyl]amino]-1-thio-L-threo-alpha-D-galacto-octopyranoside), 7-chloro-6,7,8-trideoxy-6-[[(1-methyl-4-propyl-2-pyrrolidinyl) carbonyl]-amino]-1-thio-L-threo-alpha-D-galacto-octopyranoside, (4-(dimethylamino)-1,4,4-alpha,5,5-alpha, 6,11,12-alpha-octahydro-3,6,12,12-alpha-pentahydroxy-6-methyl-1,11-dioxo-2-naphthacene-carboxamide), chlortetracycline, demeclocycline, rolitetracycline, sulfacetamide, sulfabenzamide, sulfadiazine, sulfadoxine, sulfamerazine, sulfamethazine, sulfamethizole, sulfamethoxazole, sulfacetamide sodium, amphotericin B, benzoic acid, butenafine, butenafine HCl, butoconazole, butoconazole nitrate, caprylic acid, chloroxylenol, ciclopirox, clotrimazole, econazole, econazole nitrate, fluconazole, itraconazole, ketoconazole, miconazole, miconazole nitrate, naftifine, naftifine hydrochloride, nystatin, oxiconazole, oxiconazole nitrate, salicylic acid, selenium, selenium sulfide, sulconazole, sulconazole nitrate, terbinafine, terbinafine hydrochloride, terconazole, tioconazole, undecylenic acid, acitretin, alclometasone dipropionate, anthralin, azathioprine, calcipotriene, calcitriol, colchicine, cyclosporine, methoxsalen, retinoids, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, azelaic acid, arachidonic acid, benzethonium chloride, benzalkonium chloride, boric acid, 8-quinolinol benzoate, secondary amyltricresols, cetylpyridinium chloride, chlorothymol, and 8-hydroxyquinoline sulfate, pharmaceutically acceptable salts thereof, and mixtures thereof.

Non-limiting examples of sunscreen agents useful in this regard include 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropy dibenzoylmethane, 3-benzylidene camphor, 3-(4-methylbenzylidene) camphor, titanium dioxide, zinc oxide, silica, iron oxide, 4-N,N-(2-ethylhexyl)methylaminobenzoic acid ester of 2,4-dihydroxybenzophenone, 4-N,N-(2-ethylhexyl)-methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, dihydroxycinnamic acid, trihydroxy-cinnamic acid, diphenylbutadiene, stilbene, dibenzalacetone, benzalacetophenone, naphtholsulfonates, 2-naphthol-3,6-disulfonic, 2-naphthol-6,8-disulfonic acids, di-hydroxynaphthoic acid, o- and p-hydroxybiphenyldisulfonates, coumarin, diazoles, 2-acetyl-3-bromoindazole, phenyl benzoxazole, methyl naphthoxazole, various aryl benzothiazoles, quinine salts, quinoline derivatives, 8-hydroxyquinoline, 2-phenylquinoline, hydroxy- and methoxy-substituted benzophenones, uric, violuric acids, tannic acid, hydroquinone, benzophenones, oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone, 4-isopropyldibenzoylmethane, butylmethoxydibenzoylmethane, etocrylene, octocrylene, 3-(4′-methylbenzylidene boman-2-one), terephthalylidene dicamphor sulfonic acid, 4-isopropyl-di-benzoylmethane, butylmethoxydibenzoyl-methane, 2-hydroxy-4-methoxybenzo-phenone, 2-phenyl benzimidazole-5-sulfonic acid, octyldimethyl-p-aminobenzoicacid, octocrylene, 4-N,N-(2-ethylhexyl)methyl-aminobenzoic acid ester of 2,4-dihydroxybenzophenone, N,N-di-(2-ethylhexyl)-4-aminobenzoic acid ester with 4-hydroxydibenzoylmethane, 4-N,N-(2-ethylhexyl)-methylaminobenzoic acid ester of 4-(2-hydroxyethoxy)dibenzoylmethane, N,N-di-(2-ethylhexyl)-4-aminobenzoic acid ester of 2-hydroxy-4-(2-hydroxyethoxy) benzophenone, N,N-di-(2-ethylhexyl)-4-aminobenzoic acid ester of 4-(2-hydroxyethoxy) dibenzoylmethane, pharmaceutically acceptable salts thereof, and mixtures thereof.

Non-limiting examples of anti-wrinkle and anti-atrophy agents useful in this regard include cis and trans retinoic acid, retinol, retinyl esters, salicylic acid, sulfur-containing D and L amino acids, N-acetyl derivatives sulfur-containing D and L amino acids, N-acetyl-L-cystein, thiols, ethane thiol, alpha-hydroxy acids, glycolic acid, lactic acid, phytic acid, lipoic acid, lysophosphatidic acid, skin peel agents, phenol, pharmaceutically acceptable salts thereof, and mixtures thereof.

Non-limiting examples of anti-acne agents useful in this regard include keratolytics, salicylic acid (o-hydroxybenzoic acid), 5-octanoyl salicylic acid, resorcinol, retinoids, cis and trans retinoic acid, sulfur-containing D and L amino acids, N-acetyl sulfur-containing D and L amino acids, N-acetyl-L-cysteine, lipoic acid, sebostats, flavonoids, bile salts, scymnol sulfate, deoxycholate, cholate, adapalene, azelaic acid, benzoyl peroxide, clindamycin, clindamycin phosphate, doxycycline, erythromycin, norgestimate, organic peroxides, isotretinoin, tretinoin, sulfacetamide sodium, tazarotene, pharmaceutically acceptable salts thereof, and mixtures thereof.

Skin Protective Ingredient

The presently preferred compositions additionally comprise a skin protective ingredient as an essential component. In this regard, the present compositions preferably contain about 25% to about 65% by weight of a skin protective ingredient. The skin protective ingredient of these compositions is critical to providing their softening, smoothing, lubricating, and skin protectant features. In this regard, the skin protective ingredient functions as an emollient. Additionally, once applied to skin, the skin protective ingredient lowers the transepidermal water loss (TEWL), or migration of moisture, through the skins tissues from deeper dermal tissues. Accordingly, by lubricating the skin, the skin protective ingredient of the present preferred compositions lowers the amount of TEWL experienced, thus alleviating and preventing further adverse skin disorders, such as pruritus.

The presently preferred compositions can contain one or more skin protective ingredients. In a preferred embodiment, the present compositions contain a skin protective ingredient comprising at least one C₇ or greater saturated or unsaturated, branched or unbranched, hydrocarbon chain.

Preferred skin protective ingredients useful herein generally have low solubility in water, such that preferably less than about 10% by weight is soluble in water at 25° C., and more preferably less than about 1% by weight is soluble in water at 25° C. Additionally, the skin protective ingredients useful herein preferably have a density of about 0.75 to about 1.65.

Preferred non-limiting examples of skin protective ingredients useful in the present compositions include petrolatum, red petrolatum, white petrolatum, liquid petrolatum, semi-solid petrolatum, light mineral oil, heavy mineral oil, white mineral oil, mineral oil alcohols, C₇-C₄₀ branched chain hydrocarbons, C₁₀-C₃₀ alcohol esters of C₁₀-C₃₀ carboxylic acids, C₁₀-C₃₀ alcohol esters of C₁₀-C₃₀ dicarboxylic acids, monoglycerides of C₁₀-C₃₀ carboxylic acids, diglycerides of C₁₀-C₃₀ carboxylic acids, triglycerides of C₁₀-C₃₀ carboxylic acids, ethylene glycol monoesters of C₁₀-C₃₀ carboxylic acids, ethylene glycol diesters of C₁₀-C₃₀ carboxylic acids, propylene glycol monoesters of C₁₀-C₃₀ carboxylic acids, propylene glycol diesters of C₁₀-C₃₀ carboxylic acids, C₁₀-C₃₀ carboxylic acid monesters and polyesters of sugars, polyorganosiloxanes, polydialkylsiloxanes, polydiarylsiloxanes, polyalkarylsiloxanes, cyclomethicones having 3 to 9 silicon atoms, vegetable oils, hydrogenated vegetable oils, olive oil, hydrogenated olive oil, shea butter, polypropylene glycols, polypropylene glycol C₄-C₂₀ alkyl ethers, di C₈-C₃₀ alkyl ethers, synthetic hydrocarbons, derivatives thereof, and mixtures thereof.

In a particularly preferred embodiment, the skin protective ingredient is petrolatum.

Petrolatum, which is also known as petroleum jelly, and its derivatives are colloidal systems of nonstraight-chain solid hydrocarbons and high-boiling liquid hydrocarbons, in which most of the liquid hydrocarbons are held inside the micelles. See The Merck Index, Thirteenth Edition, Budavari et al., Eds., Merck & Co., Inc., Rahway, N.J. (2001); Schindler, Drug. Cosmet. Ind., (1961); and the CTFA (Cosmetic, Toiletry, and Fragrance Association) International Cosmetic Ingredient Dictionary and Handbook, Tenth Edition (2004), which are incorporated by reference herein in their entirety.

In an alternative preferred embodiment, the skin protective ingredient is a straight or branched chain hydrocarbon having from about 7 to about 40 carbon atoms. Preferred, non-limiting examples of these hydrocarbon materials include dodecane, isododecane, squalane, cholesterol, hydrogenated polyisobutylene, docosane (i.e. a C₂₂ hydrocarbon), hexadecane, isohexadecane, derivatives thereof, and mixtures thereof. Also useful are the C₇-C₄₀ isoparaffins, which are C₇-C₄₀ branched hydrocarbons.

Additionally, further alternative useful skin protective ingredients for the present compositions include straight and branched chain hydrocarbons and aromatic derivatives of C₁₀-C₃₀ alcohol esters of C₁₀-C₃₀ carboxylic acids and of C₁₀-C₃₀ dicarboxylic acids, ethylene glycol monoesters of C₁₀-C₃₀ carboxylic acids, derivatives thereof, and mixtures thereof. Preferred carboxylic acids useful herein include C₁₀-C₃₀ straight chain, branched chain, and aryl carboxylic acids, as well as propoxylated and ethoxylated drivatives of these carboxylic acids. Additionally preferred, non-limiting examples of such alternative skin protective ingredients include diisopropyl sebacate, diisopropyl adipate, isopropyl myristate, isopropyl palmitate, myristyl propionate, ethylene glycol distearate, 2-ethylhexyl palmitate, isodecyl neopentanoate, C₁₂₋₁₅ alcohols benzoate, di-2-ethylhexyl maleate, cetyl palmitate, myristyl myristate, stearyl stearate, cetyl stearate, behenyl behenrate, dioctyl maleate, dioctyl sebacate, diisopropyl adipate, cetyl octanoate, diisopropyl dilinoleate, caprilic/capric triglyceride, PEG-6 caprylic/capric triglyceride, PEG-8 caprylic/capric triglyceride, derivatives thereof, and mixtures thereof.

In another alternative preferred embodiment, the skin protective ingredient is a C₁₀-C₃₀ monester or polyester of a sugar or a related material. These esters are derived from a sugar or polyol moiety and one or more carboxylic acid moieties. Depending on the constituent acid and sugar, these esters can be in either liquid or solid form at room temperature. Preferred, non-limiting examples of such liquid esters include glucose tetraoleate, glucose tetraesters of soybean oil fatty acids, mannose tetraesters of mixed soybean oil fatty acids, galactose tetraesters of oleic acid, arabinose tetraesters of linoleic acid, xylose tetralinoleate, galactose pentaoleate, sorbitol tetraoleate, sorbitol hexaesters of unsaturated soybean oil fatty acids, xylitol pentaoleate, surcrose tetraoleate, sucrose pentaoletate, sucrose hexaoleate, sucrose hepatoleate, sucrose octaoleate, derivatives thereof, and mixtures thereof.

Preferred, non-limiting examples of solid esters useful as a skin protective ingredient in the present compositions include sorbitol hexaester in which the carboxylic acid ester moieties are palmitoleate and arachidate, octaester of raffinose in which the carboyxlic acid ester moieties are linoleate and behenate, heptaester of maltose wherein the esterifying carboxylic acid moieties are sunflower seed oil fatty acids and lignocerate, octaester of sucrose wherein the esterifying carboxylic acid moieties are oleate and behanate, the octaester of sucrose wherein the esterifying carboxylic acid moieties are laurate, linoleate and behenate, derivatives thereof, and mixtures thereof. A preferred solid material is surcrose polyester in which the degree of esterification is 7-8, and in which the fatty acid moieties are C₁₈ mono- and/or di-unsaturated and behenic. Another preferred solid sugar polyester useful herein is the octaester of sucrose in which there are about 7 behenic fatty acid moieties and about 1 oleic acid moiety in the molecule.

In yet another alternative preferred embodiment, the skin protective ingredient in the present composition can be an organosilicone such as a polyalkylsilicone, a cyclic polyalkylsiloxane, a polydialkylsiloxane, a polydiarylsiloxane, a polyalkarylsiloxane, or a cyclomethicone having 3 to 9 silicon atoms, and can be volatile or nonvolatile. Preferred polyalkylsiloxanes have a viscosity of from about 0.5 to about 100,000 centistokes at 25° C., and correspond to the general chemical formula R₃SiO[R₂SiO]ₓSiR₃ wherein R₂ and R₃ are alkyl groups, while x is an integer from about 0 to about 500. Non-limiting examples of preferred polyalkylsiloxanes useful in this regard include the Vicasil® series sold by General Electric Company and the Dow Corning® 200 series sold by Dow Corning Corporation.

Additionally preferred cyclic polyalkylsiloxanes useful as skin protective ingredients in the present compositions include those corresponding to the general chemical formula [SiR₂O]ₙ wherein R₂ is an alkyl group and n is an integer from about 3 to about 9, more preferably n is an integer from about 3 to about 7, and most preferably n is an integer from about 4 to about 6. When R₂ is methyl, these materials are typically referred to as cyclomethicones. Preferred, non-limiting examples of such cyclomethicones include Dow Corning® 244 fluid, which primarily contains the cyclomethicone tetramer (i.e. n=4), Dow Corning® 344 fluid, which primarily contains the cyclomethicone pentamer (i.e. n=5), Dow Corning® 245 fluid, which primarily contains a mixture of the cyclomethicone tetramer and pentamer (i.e. n=4 and 5), Dow Corning® 345 fluid, which primarily contains a mixture of the cyclomethicone tetramer, pentamer, and hexamer (i.e. n=4, 5, and 6), derivatives thereof, and mixtures thereof.

In yet another alternative preferred embodiment, the skin protective ingredient can be a trimethylsiloxysilicate, which is a polymeric material corresponding to the general chemical formula [(CH₂)₃]ₓ [SiO₂]_{y}, wherein x is an integer from about 1 to about 500 and y is an integer from about 1 to about 500. A preferred, non-limiting example of a useful trimethylsiloxysilicate in this regard is Dow Corning® 593 fluid.

Other preferred skin protective ingredients in this regard include dimethiconols, which are hydroxy terminated dimethyl silicones, represented by the general chemical formulas R₅SiO[R₄SiO]ₓSiR₄OH and HOR₄SiO[R₄SiO]ₓSiR₄OH wherein R₄ and R₅ are an alkyl group (preferably R₄ is methyl or ethyl, more preferably methyl) and x is an integer from 0 to about 500. Preferred, non-limiting examples of dimethiconols in this regard include mixtures with dimethicone or cyclomethicone, such as but not limited to, Dow Corning® 1401, 1402, and 1403 fluids.

In another alternative preferred embodiment, the skin protective ingredient is a polyalkylaryl siloxane, which includes polymethyphenyl siloxane, such as SF 1075 methylphenyl fluid sold by General Electric Company and 556 Cosmetic Grade phenyl trimethicone fluid sold by Dow Corning Corporation.

In additional alternative preferred embodiments, vegetable oils and hydrogenated vegetable oils can be used as skin protective ingredients in the present compositions. Preferred, non-limiting examples of vegetable oils and hydrogenated vegetable oils useful in this regard include safflower oil, caster oil, coconut oil, cottenseed oil, menhaden oil, palm kernel oil, palm oil, peanut oil, soybean oil, rapeseed oil, linseed oil, rice bran oil, pine oil, sesame oil, sunflower seed oil, hydrogenated safflower oil, hydrogenated caster oil, hydrogenated coconut oil, hydrogenated cottenseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, olea europaea oil, hydrogenated olea europaea oil, palm glycerides, hydrogenated palm glycerides, derivatives thereof, and mixtures thereof.

In yet another alternative preferred embodiment, the skin protective ingredient can be a polyproylene glycol, a C₄-C₂₀ alkyl ether of polypropylene glycol, a C₁-C₂₀ carboxylic acid ester of polypropylene glycol, a di-C₈-C₃₀ alkyl ether, a derivative thereof, or a mixture thereof. Preferred, non-limiting examples of such materials include PPG-14 butyl ether, PPG-15 stearyl ether, PPG-9, PPG-12, PPG-15, PPG-17, PPG-20, PPG-26, PPG-30, PPG-34, dioctyl ether, dodecyl octyl ether, derivatives thereof, and mixtures thereof.

In a most preferred embodiment, the skin protective ingredient is selected from the group consisting of petrolatum, red petrolatum, white petrolatum, liquid petrolatum, semi-solid petrolatum, light mineral oil, heavy mineral oil, white mineral oil, mineral oil alcohols, C₇-C₄₀ branched chain hydrocarbons, derivatives thereof, and mixtures thereof.

Oleaginous Solvent

The presently preferred compositions further comprise an oleaginous solvent comprising a substance other than the skin protective ingredient. The oleaginous solvent component provides a medium to dissolve and evenly disperse the skin protective ingredient therein. The oleaginous solvent also provides extra protection against excess water loss from the stratum corneum and undesirable skin conditions affected by excessive TEWL. The oleaginous solvents useful herein can comprise a single component or a combination of components, can be a liquid or semi-liquid at about 25° C., can be saturated or unsaturated, and can be branched or unbranched.

In a preferred embodiment, the oleaginous solvent is a fatty alcohol, a fatty acid, a fatty ester, a fatty ether, derivatives thereof, or mixtures thereof. Additionally, the oleaginous solvent is preferably present in the instant compositions in an amount of at least about 4% by weight. In a more preferred embodiment, the oleaginous solvent is present in the instant compositions in an amount of at least about 6% by weight, and in a most preferred embodiment the oleaginous solvent is present in an amount of at least about 8% by weight. However, it is important that the oleaginous solvent is not present in an amount sufficient to render the present compositions greasy, such as is typical for many ointments.

Preferred fatty alcohols and fatty acids useful in the present compositions include those having from about 10 to about 30 carbon atoms, preferably from about 12 to about 28 carbon atoms, and more preferably from about 16 to about 24 carbon atoms. Additionally, the preferred fatty alcohols and fatty acids may be straight or branched chain alcohols and may be saturated or unsaturated alcohols. In a preferred embodiment, the oleaginous solvent is a liquid fatty alcohol or liquid fatty acid. Preferred, non-limiting examples of liquid fatty alcohols and liquid fatty acids useful in this regard include oleyl alcohol, palmitoleic alcohol, isostearyl alcohol, isocetyl alcohol, oleic acid, linoleic acid, isostearic acid, linolenic acid, ethyl linolenic acid, ethyl linolenic acid, arachidonic acid, ricinolic acid, derivatives thereof, and mixtures thereof.

Additionally preferred fatty acid esters and fatty acid ethers useful in the present compositions include, but are not limited to, esters of fatty acids, alkoxylated fatty alcohols, alkyl ethers of fatty alcohols, alkyl ethers of alkoxylated fatty alcohols, derivatives thereof, and mixtures thereof. Preferred, non-limiting examples of fatty acid esters and fatty acid ethers useful in this regard include polyoxyethylene sorbitan monooleate, polysorbate 80, methyl linoleate, ethyl linoleate, isopropyl linoleate, isodecyl oleate, isopropyl oleate, ethyl oleate, octyldodecyl oleate, oleyl oleate, decyl oleate, butyl oleate, methyl oleate, octyldodecyl stearate, octyldodecyl isostearate, octyldodecyl isopalmitate, octyl isopelargonate, octyl pelargonate, hexyl isostearate, isopropyl isostearate, isodecyl isononanoate, isopropyl isostearate, ethyl isostearate, methyl isostearate, oleth-2, derivatives thereof, and mixtures thereof.

In a more preferred embodiment, the oleaginous solvent is ethyl oleate or a derivative thereof.

Aqueous Solvent

The presently preferred compositions additionally comprise an aqueous solvent. In a preferred embodiment, the present compositions comprise about 10% to about 90% by weight of the aqueous solvent. In a more preferred embodiment, the present compositions comprise about 15% to about 80% by weight of the aqueous solvent. In a most preferred embodiment, the present compositions comprise about 35% to about 75% by weight of the aqueous solvent.

Dermatologically Acceptable Excipients

The preferred compositions discussed herein can additionally comprise at least one dermatologically acceptable excipient commonly known to those of ordinary skill in the art as useful in topical compositions. Preferred, non-limiting examples of dermatologically acceptable excipients useful in these compositions are those selected from the group consisting of moisturizers, preservatives, gelling agents, colorants or pigments, antioxidants, radical scavengers, surfactants, emulsifiers, pH modifiers, chelating agents, derivatives thereof, and mixtures thereof.

Moisturizers

The presently preferred compositions may optionally further contain a moisturizer. Preferred non-limiting examples of moisturizers that can optionally be included in these compositions include glycerin, pentylene glycol, butylene glycol, polyethylene glycol, sodium pyrrolidone carboxylate, alpha-hydroxy acids, beta-hydroxy acids, polyhydric alcohols, ethoxylated and propoxylated polyols, polyols, polysaccharides, panthenol, hexylene glycol, propylene glycol, dipropylene glycol, sorbitol, derivatives thereof, and mixtures thereof.

Preservatives

The presently preferred compositions may optionally further contain a preservative. Preferred non-limiting examples of preservatives that can optionally be included in these compositions include propylene glycol, glycerol, butylene glycol, pentylene glycol, hexylene glycol, sorbitol, benzyl alcohol, derivatives thereof, and mixtures thereof.

A particularly preferred preservative in this regard is benzyl alcohol or a derivative thereof. Additionally, the preservative is preferably present in an amount of about 0.1% to about 2.5% by weight of the overall weight of the composition.

Gelling Agents

The presently preferred compositions may optionally further contain a gelling agent. Preferred non-limiting examples of gelling agents that can optionally be included in these compositions include various cellulose agents, hydroxyethylcellulose, xanthan gum, sodium carbomer, carbomer, polyacrylic polymers, derivatives thereof, and mixtures thereof. Other suitable gelling agents which may be useful in the present compositions include aqueous gelling agents, such as neutral, anionic, and cationic polymers, derivatives thereof, and mixtures thereof.

Exemplary polymers which may be useful in the preferred compositions include carboxy vinyl polymers, such as carboxypolymethylene. In this regard, a preferred gelling agent is a Carbopol® polymer (i.e. a polyacrylic polymer) such as is available from Noveon Inc., Cleveland, Ohio. Another preferred gelling agent is a Pemulen® polymer (i.e. a polyacrylic polymer) such as is available from Noveon Inc., Cleveland, Ohio.

Other suitable gelling agents useful in the present compositions include cellulosic polymers, such as gum arabic, gum tragacanth, locust bean gum, guar gum, xanthan gum, cellulose gum, methylcellulose, hydroxyethylcellulose, hydroxypropylcellulose, hydroxypropylmethylcellulose, derivatives thereof, and mixtures thereof.

The gelling agent is preferably present in the instant compositions in an amount of from about 0.01% to about 10%, more preferably from about 0.1% to about 5%, and most preferably from about 0.1% to about 2%, by weight.

Anti-Oxidants

The presently preferred compositions may optionally further contain an anti-oxidant. Preferred non-limiting examples of anti-oxidants that can optionally be included in these compositions include ascorbic acid, ascorbyl esters of fatty acids, magnesium ascorbyl phosphate, sodium ascorbyl phosphate, ascorbyl sorbate, tocopherol, tocopherol sorbate, tocopherol acetate, butylated hydroxy benzoic acid, thioglycolates, persulfate salts, 6-hydroxy-2,5,7,8-tetramethylchroman-2-carboxylic acid, lipoic acid, gallic acid, propyl gallate, uric acid, sorbic acid, lipoic acid, amines, N,N-diethylhydroxylamine, N-acetyl-L-cysteine, amino-guanidine, sulfhydryl compounds, glutathione, dihydroxy fumaric acid, lycine pidolate, arginine pilolate, nordihydroguaiaretic acid, bioflavonoids, curcumin, lysine, 1-methionine, praline, superoxide dismutase, silymarin, tea extracts, grape skin/seed extracts, melanin, rosemary extracts, derivatives thereof, and mixtures thereof.

Surfactants

The presently preferred compositions may optionally further contain a surfactant. Preferred non-limiting examples of surfactants that can optionally be included in these compositions include zwitterionic, amphoteric, anionic, cationic, nonionic, and mixtures thereof. Preferred zwitterionic, amphoteric, anionic, cationic, and nonionic surfactants include those disclosed in McCutcheon's, Detergents and Emulsifiers, North American edition (1986), published by allured Publishing Corporation, and McCutcheon's, Functional Materials, North American Edition (1992), both of which are incorporated by reference herein in their entirety.

Emulsifiers

The presently preferred compositions may optionally further contain an emulsifier. Preferred non-limiting examples of emuslifiers that can optionally be included in these compositions include any of a wide variety of nonionic, cationic, anionic, zwitterionic and amphoteric emulsifiers.

Preferred, non-limiting examples of specific emulsifiers useful in this regard include glycol esters, fatty acids, fatty alcohols, fatty acid glycol esters, fatty esters, fatty ethers, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, derivatives thereof, and mixtures thereof.

In a preferred embodiment, the present compositions can comprise about 0.3% to about 15% by weight of an emulsifier. In a more preferred embodiment, the present compositions can comprise about 3% to about 10% by weight of an emulsifier.

pH Modifiers

The presently preferred compositions may optionally further contain a pH modifier. Preferred non-limiting examples of neutralizing pH modifiers that can optionally be included in these compositions include inorganic hydroxides, inorganic oxides, inorganic salts of weak acids, derivatives thereof, and mixtures thereof.

Preferred, non-limiting examples of inorganic hydroxides useful in this regard include ammonium hydroxide, alkali metal hydroxide, alkaline earth metal hydroxides, derivatives thereof, and mixtures thereof.

Preferred inorganic hydroxides useful in this regard include ammonium hydroxide, monovalent alkali metal hydroxides such as sodium hydroxide and potassium hydroxide, divalent alkali earth metal hydroxides such as calcium hydroxide and magnesium hydroxide, derivatives thereof, and mixtures thereof.

Preferred, non-limiting examples of inorganic oxides useful in this regard include magnesium oxide, calcium oxide, derivatives thereof, and mixtures thereof.

Preferred, non-limiting examples of inorganic salts of weak acids useful in this regard include ammonium phosphate (dibasic), alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic), alkaline earth metal salts of weak acids such as magnesium phosphate and calcium phosphate, derivatives thereof, and mixtures thereof.

Chelating Agents

The presently preferred compositions may optionally further contain a chelating agent. Preferred non-limiting examples of chelating agents that can optionally be included in these compositions include citric acid, isopropyl (mono) citrate, stearyl citrate, lecithin citrate, gluconic acid, tartaric acid, oxalic acid, phosphoric acid, sodium tetrapyrophosphate, potassium monophosphate, sodium hexametaphosphate, calcium hexametaphosphate, sorbitol, glycine (aminoacetic acid), methyl glucamine, triethanolamine (trolamine), EDTA, DEG (dihydroxyethylglycine), DPTA (diethylene triamine pentaacetic acid), NTA (Nitrilotriacetic Acid), HEDTA (N-(hydroxyethyl)-ethylenetriaminetriacetic acid), aminocarboxylates, dimercaperol (BAL), larixinic acid (Maltol), unidentate ligands (fluoride and cyanide ions), diphenylthiocarbazone, 0-phenanthroline, barium diphenylamine sulfonate, sodium glucoheptonate, 8-hydroxyquinoline, olefin complexes (such as dicyclopentadienyl iron), porphyrins, phosponates, pharmaceutically acceptable salts thereof, derivatives thereof, and mixtures thereof.

In addition to those enumerated above, any other pharmaceutically active agent, skin protective ingredient, oleaginous solvent, moisturizer, preservative, gelling agent, colorant or pigment, antioxidant, radical scavenger, surfactant, emulsifier, pH modifier, chelating agent, or other dermatologically acceptable excipient commonly known to those of ordinary skill in the art as useful in topical compositions is contemplated as useful in the compositions described herein. Further, any non-toxic, inert, and effective topical carrier may be used to formulate the compositions described herein. Well-known carriers used to formulate other topical therapeutic compositions for administration to humans will be useful in these compositions. Examples of these components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, January 1996, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

In another particularly preferred embodiment, the presently preferred pharmaceutical compositions are formulated in a lotion, cream, ointment, gel, suspension, emulsion, foam, aerosol, or other pharmaceutically acceptable topical dosage form.

### Methods of Treatment

Another preferred aspect of the present subject matter pertains to a method for inhibiting or treating a pruritic skin condition in a patient, which comprises administering to a patient in need thereof a topical composition comprising:

(i) a therapeutically effective amount of a pharmaceutically active agent comprising an anesthetic agent or a derivative thereof;

(ii) about 25 to about 65% by weight of a skin protective ingredient;

(iii) an oleaginous solvent comprising a substance other than said skin protective ingredient; and

(iv) an aqueous solvent;

wherein said skin protective ingredient enhances the ability of the pharmaceutically active agent to inhibit or treat the pruritic skin condition.

In this regard, the preferred compositions described herein can be used in methods for temporarily or permanently reducing, inhibiting, treating, ameliorating, or preventing pruritic skin conditions, as well as other skin disorders, and restoring or repairing a skin lipid barrier of a mammal.

These methods can be achieved by topically applying the presently preferred compositions to the skin of a patient, such as a mammal. The skin protective ingredient of the present compositions functions as an emollient, lubricating the stratum corneum and lowering the amount of TEWL experienced by the skin. All of these functions allow the present compositions to temporarily or permanently alleviate pruritus and/or other skin disorders experienced by the mammal. Additionally, the pharmaceutically active agent helps alleviate itchy, burning, irritated sensations caused by the skin disorders, such as pruritus.

The presently preferred compositions can further fortify the skin lipid barrier to prevent its disruption due to environmental insults. In this regard, once topically applied to the skin of a mammal, the preferred compositions lubricate the stratum corneum, increase intercellular adhesion in the skin of the mammal, and lower the TEWL experienced by the skin. This increased intercellular adhesion results in the restoration and/or repair of the skin lipid barrier.

This repair of the skin lipid barrier improves the skin barrier function and conveys numerous additional therapeutic effects to a mammal to which the preferred compositions are applied. For example, this skin lipid barrier repair can further enhance the repair of the skin to which the compositions are applied, increase the interstitial oil content of the skin, improve the integrity of the skin's interstitial lipid layer, treat skin disorders such as pruritus, and reduce the occurrence of further skin barrier malfunctions. The increased interstitial oil content of the skin and the improved integrity of the skin's interstitial lipid layer is a direct result from the enhanced skin repair. Accordingly, the present skin protectant compositions are unexpectedly useful in methods of treating any mammalian skin areas.

The improved skin barrier function is a result of the unique pH characteristics of the present compositions. The specific, narrow pH of the present compositions, i.e. a pH of about 6 to about 8, has a significant impact upon application to the skin. In particular, the present compositions have the unique ability to normalize the pH of the skin to a predetermined optimal skin pH. This normalized skin pH results in an improved skin barrier function.

In addition to and concurrently with the skin repair, the increased intercellular adhesion resulting from administration of the present compositions further reduces manifestations of pruritus while enhancing the skin repair. This reduction of pruritus manifestations is optimally achieved by daily topically applying the preferred compositions to the skin of a mammal. These compositions are superior to those compositions presently available for the reduction of pruritus, and thus for the moisturization of the skin, due to their extended therapeutic characteristics. Accordingly, the presently preferred compositions provide both an immediate therapeutic effect, as well as an extended therapeutic effect.

In an alternative embodiment, non-limiting examples of additional skin conditions potentially treatable with the present skin protectant compositions include atopic dermatitis, itching, eczema, ichthyosis, psoriasis, seborrheic dermatitis, eczematous dermatitis, ulcers and erosions due to cutaneous trauma, epidermolysis bullosa, cutaneous changes of intrinsic or extrinsic aging, and combinations thereof.

In an especially preferred embodiment, the present subject matter further contemplates reducing the incidence of further occurrences of these skin conditions, including pruritis, in addition to the initial treatment.

Combination Therapy

In another preferred embodiment, the present preferred compositions may be used in combination with an additional pharmaceutical dosage form to enhance their effectiveness in treating a dermatological disease or disorder. In this regard, the present preferred compositions may be administered as part of a regimen additionally including any other pharmaceutical and/or pharmaceutical dosage form known in the art as effective for the treatment of a dermatological disorder. Similarly, a pharmaceutically active ingredient other than those specified herein can be added to the present preferred compositions to enhance their effectiveness in treating a dermatological disease or disorder. Accordingly, this additional pharmaceutically active ingredient or additional pharmaceutical dosage form can be applied to a patient either directly or indirectly, and concomitantly or sequentially, with the preferred compositions described herein.

In one embodiment in this regard, the present preferred composition and the additional pharmaceutical dosage form can be administered to a patient at the same time. In an alternative embodiment, one of the present preferred compositions and the additional pharmaceutical dosage form can be administered in the morning and the other can be administered in the evening.

### Methods of Production

Another preferred aspect relates to a process for preparing a composition suitable for topical administration, said process comprising:

1) preparing an oil phase comprising about 25% to about 65% by weight of the overall weight of the composition of a skin protective ingredient and an oleaginous solvent comprising a substance other than the skin protective ingredient, and heating to a temperature about 75 to about 85° C.;

2) preparing an aqueous phase comprising about 35% to about 75% of the overall weight of the composition of water and a gelling agent, and heating to a temperature of about 75 to about 85° C.;

3) adding said aqueous phase to said oil phase while stirring at a temperature of about 75 to about 85° C. to obtain an emulsion;

4) cooling said emulsion to a temperature of about 55 to about 65° C.;

5) adding a pramoxine solution to said emulsion;

6) adding a sodium hydroxide solution to said emulsion to obtain an emulsion having a pH of about 6 to about 8;

7) adding a benzyl alcohol solution to said emulsion; and

8) recovering a topical pharmaceutical composition.

In another preferred embodiment, the oil phase is prepared by first mixing the skin protective ingredient and the at least one oleaginous solvent before the addition of the aqueous phase. In a preferred embodiment, the skin protective ingredient is pre-heated and mixed with the at least one oleaginous solvent under high stirring.

In a further preferred embodiment, the aqueous phase is prepared by first mixing the gelling agent before adding it to the oil phase. In this regard, the gelling agent is preferably added to the aqueous phase while heating the aqueous phase to a temperature of about 75 to about 85° C. under high stirring. In a particularly preferred embodiment, the gelling agent is selected from the group consisting of xanthan gum, carbomer, sodium carbomer, a polyacrylic polymer, and mixtures thereof.

In another preferred embodiment, the aqueous phase is mixed after the first oleaginous solvent is added until the skin protective ingredient is completely dissolved and evenly dispersed in the emulsion.

The present processes preferably form compositions comprising an emulsion having an oil phase and an aqueous phase. Non-limiting examples of specific types of emulsions that can be made according to this process include an oil-in-water emulsion, a water-in-oil emulsion, an oil-in-water-in-oil emulsion, and a water-in-oil-in-water emulsion. The formation of a specific type of emulsion will depend on the specific ingredients used in the process. In a preferred embodiment, the process will form compositions that are oil-in-water emulsions.

This particular preparation process is a non-limiting example of a possible process that can be used to prepare the preferred compositions. Other processes capable of preparing these compositions are further contemplated herein. Further, the individual phases of the preferred compositions (for example aqueous and oil phases) can be prepared sequentially in any order or concurrently; it is not necessary to prepare the oil phase before the aqueous phase is prepared in order to practice the present processes. Additionally, preferred compositions can be prepared according to either a batch process or continuously.

Further contemplated as within the scope of the present subject matter are pharmaceutical compositions produced according to the above-described process. If produced according to this process, these compositions exhibit chemical and physical stability suitable for topical administration.

The compositions produced according to these processes can be placed in a suitable containment vessel comprising a product contact surface composed of a material selected from the group consisting of glass, plastic, steel, stainless steel, aluminum, Teflon, polymeric structure, ceramic structure, alloys, and mixtures thereof. These containment vessels are used to facilitate manufacturing, handling, processing, packaging, storage, and administration of said composition. Preferred containment vessels in this regard can be selected from the group consisting of plastic tubes, bottles, metal tubes, and any combination thereof.

### Dosage

Appropriate dosage levels for the pharmaceutically active agents contemplated in the preferred compositions and methods are well known to those of ordinary skill in the art and are selected to maximize the treatment of the above skin conditions. Dosage levels on the order of about 0.001 mg to about 5,000 mg per kilogram body weight of the skin protective ingredient and pharmaceutically active agent components are known to be useful in the treatment of the diseases, disorders, and conditions contemplated herein. Typically, this effective amount of the skin protective ingredient and pharmaceutically active agent will generally comprise from about 0.001 mg to about 100 mg per kilogram of patient body weight per day. Moreover, it will be understood that this dosage of ingredients can be administered in a single or multiple dosage units to provide the desired therapeutic effect.

If desired, other therapeutic agents can be employed in conjunction with those provided in the above-described compositions. The amount of pharmaceutically active ingredients that may be combined with the carrier materials to produce a single dosage form will vary depending upon the host treated, the nature of the disease, disorder, or condition, and the nature of the active ingredients.

The preferred pharmaceutical compositions may be given in a single or multiple doses daily. In a preferred embodiment, the pharmaceutical compositions are given from one to three times daily. Starting with a low dose twice daily and slowly working up to higher doses if needed is a preferred strategy. The amount of pharmaceutically active ingredients that may be combined with the carrier materials to produce a single dosage form will vary depending upon the host treated, the nature of the disease, disorder, or condition, and the nature of the active ingredients.

It is understood, however, that a specific dose level for any particular patient will vary depending upon a variety of factors, including the activity of the specific skin protective ingredient and pharmaceutically active agent; the age, body weight, general health, sex and diet of the patient; the time of administration; the rate of excretion; possible drug combinations; the severity of the particular condition being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular skin protective ingredient and pharmaceutically active agent combination and the desired dosage. See, for example, “Remington's Pharmaceutical Sciences” , 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the essential lipids.

### EXAMPLES

The following examples are illustrative of preferred compositions and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates the preparation of a present preferred cream:

| | % W/W |
|---|---|
| Purified Water | 51.81 |
| Carbomer | 0.5 |
| White Petrolatum, USP | 30.0 |
| Ethyl Oleate | 10.0 |
| Stearic Acid | 2.0 |
| PEG-8 Stearate | 2.0 |
| Glyceryl Stearate & PEG-100 Stearate | 1.0 |
| Sodium Hydroxide | 0.4 |
| Pramoxine Hydrochloride, USP | 1.0 |
| Benzyl Alcohol | 0.7 |
| Menthol, USP | 0.59 |
| | 100.0% |

Preparation of the Cream:

1. An oil phase is prepared by preheating the petrolatum to 50±5° C. After the petrolatum has been pre-heated, the petrolatum is mixed with the ethyl oleate, stearic acid, PEG-8 stearate, glyceryl stearate & PEG-100 stearate at 80±20° C. until all ingredients are melted and a uniform appearance is produced.

2. An aqueous phase is prepared by heating and mixing about 400 kg of purified water to 80±2° C. The Carbomer is then added to the water and mixed for about 40 minutes at 80±2° C., or until the mixture is homogenized.

3. The oil phase is then placed in a vacuum at 500±50 mbar and is mixed at 80±2° C. While mixing the oil phase in the vacuum at the given pressure and temperature, the aqueous phase is added to the oil phase and is mixed for about 15 minutes while the mixture of the oil and aqueous phases is maintained at 80±2° C. The mixture is then reduced in temperature to 60±2° C. while being maintained in the vacuum at 500±50 mbar.

4. A pramoxine hydrochloride solution is prepared by adding pramoxine hydrochloride to about 27.5 kg of purified water while mixing at 1100±200 rpm. The solution of water and pramoxine is then mixed for about 20 minutes, or until the pramoxine is dissolved.

5. While mixing, the pramoxine solution is added to the oil and aqueous phase mixture. Any additionally needed purified water is added. This mixture is then mixed and maintained in the vacuum at 500±50 mbar at 60±2° C. for about 15 minutes.

6. A sodium hydroxide solution is prepared by mixing purified water with the sodium hydroxide. While mixing, the sodium hydroxide solution is then added to the mixture of the aqueous and oil phases and pramoxine solution. This mixture is then mixed and maintained in the vacuum at 500±50 mbar at 60±2° C. for about 15 minutes, after which the mixture is reduced in temperature to 35±2° C. while continuing to stir in the vacuum at 500±50 mbar.

7. A benzyl alcohol and menthol phase is prepared by mixing benzyl alcohol and menthol together for about 10 minutes, or until the phase is homogenized. The benzyl alcohol and menthol phase is then added while mixing to the mixture of the aqueous and oil phases, pramoxine solution, and hydroxide solution. This mixture is continuously mixed at 35±2° C. for about 20 minutes.

8. This final mixture is then tested to ensure the pH is about 7.0 to about 7.6. If the mixture has a pH lower than about 7.0, the solution is brought to a pH of about 7.0 to about 7.6 with a sodium hydroxide solution. If the mixture has a pH higher than about 7.6, the solution is brought within the pH of about 7.0 to about 7.6 with an appropriate acidic solution.

### Example 2

A patient is suffering from pruritus. A preferred composition herein is topically administered to the patient. It would be expected that the patient would improve his/her condition or recover.

### Example 3

A patient is suffering from a damaged skin lipid barrier. A preferred composition herein is topically administered to the patient. It would be expected that the patient would improve his/her condition or recover.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

What is claimed is:

1. A topical skin protectant composition comprising:
(i) a therapeutically effective amount of a pharmaceutically active agent comprising an anesthetic agent or a derivative thereof;
(ii) about 25 to about 65% by weight of a skin protective ingredient;
(iii) an oleaginous solvent comprising a substance other than said skin protective ingredient; and
(iv) an aqueous solvent.

2. The composition of claim 1, wherein said anesthetic agent is selected from the group consisting of pramoxine, pramocaine, proxazocain, 4-(3-(p-butoxyphenoxy)propyl) morpholine, gamma-morpholinopropyl 4-n-butoxyphenyl ether, p-butoxyphenyl gamma-morpholinopropyl ether, 4-[3-(4-butoxyphenoxy)-propyl]morpholine, pharmaceutically acceptable salts thereof, and mixtures thereof.

3. The composition of claim 1, comprising about 0.01 to about 5% by weight of said pharmaceutically active agent.

4. The composition of claim 1, wherein said skin protective ingredient has a density of about 0.75 to about 1.65.

5. The composition of claim 1, wherein said skin protective ingredient is petrolatum or a derivative thereof.

6. The composition of claim 1, comprising at least about 8% by weight of said oleaginous solvent.

7. The composition of claim 6, wherein said oleaginous solvent is selected from the group consisting of a fatty ester, a fatty alcohol, a fatty acid, a fatty ether, derivatives thereof, and mixtures thereof.

8. The composition of claim 1, further comprising a polyacrylic polymer.

9. The composition of claim 1, further comprising an anti-pruritic agent.

10. The composition of claim 9, wherein said anti-pruritic agent is selected from the group consisting of menthol, camphor, phenol, methyl anthranilate, menthyl anthranilate, derivatives thereof, and mixture thereof.

11. The composition of claim 1, comprising about 15 to about 80% by weight of said aqueous solvent.

12. The composition of claim 1, wherein said composition further comprises a dermatologically acceptable excipient selected from the group consisting of a moisturizer, a preservative, a gelling agent, a colorant or a pigment, an antioxidant, a radical scavenger, a surfactant, an emulsifier, a pH modifier, a chelating agent, derivatives thereof, and mixtures thereof.

13. The composition of claim 1, wherein said composition is selected from the group consisting of a gel, cream, lotion, suspension, emulsion, ointment, foam, aerosol, and mixtures thereof.

14. A topical emulsion composition comprising:
(i) about 0.01 to about 5% by weight of a pharmaceutically active agent comprising an anesthetic agent or a derivative thereof;
(ii) about 25 to about 65% by weight of a skin protective ingredient;
(iii) about 0.1 to about 10% by weight of an anti-pruritic agent;
(iv) at least about 8% by weight of an oleaginous solvent comprising a substance other than said skin protective ingredient; and
(v) about 35 to about 75% by weight of an aqueous solvent.

15. The composition of claim 14, wherein said anesthetic agent is selected from the group consisting of pramoxine, pramocaine, proxazocain, 4-(3-(p-butoxyphenoxy)propyl) morpholine, gamma-morpholinopropyl 4-n-butoxyphenyl ether, p-butoxyphenyl gamma-morpholinopropyl ether, 4-[3-(4-butoxyphenoxy)-propyl]morpholine, pharmaceutically acceptable salts thereof, and mixtures thereof.

16. The composition of claim 14, wherein said oleaginous solvent is selected from the group consisting of a fatty ester, a fatty alcohol, a fatty acid, a fatty ether, derivatives thereof, and mixtures thereof.

17. The composition of claim 14, wherein said anti-pruritic agent is selected from the group consisting of menthol, camphor, phenol, methyl anthranilate, menthyl anthranilate, derivatives thereof, and mixtures thereof.

18. A method for inhibiting or treating a pruritic skin condition in a patient, which comprises:
administering to a patient in need thereof a topical composition comprising:
(i) a therapeutically effective amount of a pharmaceutically active agent comprising an anesthetic agent or a derivative thereof;
(ii) about 25 to about 65% by weight of a skin protective ingredient;
(iii) an oleaginous solvent comprising a substance other than said skin protective ingredient; and
(iv) an aqueous solvent;
wherein said skin protective ingredient enhances the ability of said pharmaceutically active agent to inhibit or treat said pruritic skin condition.

19. The method of claim 18, wherein said administering step is conducted using direct or indirect administration.

20. The method of claim 18, wherein said topical composition is administered in conjunction with another therapeutic composition effective for inhibiting or treating said pruritic skin condition.

21. The method of claim 20, wherein said other therapeutic composition is administered either concomitantly or sequentially with said topical composition.

22. The method of claim 18, wherein said topical composition further comprises an anti-pruritic agent which provides further skin protection.
